(19) United States
(12) Patent Application Publication
(10) Pub. No.: US 2007/0125663 A1
(43) Pub. Date: Jun. 7, 2007

(54) Liquid-condition detection element and detection sensor

(76) Inventors: Takeo Sasanuma, Komaki-shi (JP); Yoshikuni Sato, Komaki-shi (JP); Yuki Mizuno, Komaki-shi (JP)

Correspondence Address: STITES & HARBISON PLLC, 1199 NORTH FAIRFAX STREET, SUITE 900, ALEXANDRIA, VA, 22314, US

(21) Appl. No.: 11/605,257

(22) Filed: Nov. 29, 2006

(30) Foreign Application Priority Data
Dec. 7, 2005 (JP) JP 2005-353736

(51) Int. Cl. G01N 33/18; G01N 17/04

(52) U.S. Cl. 205/777; 436/39

(57) ABSTRACT

A liquid-condition detection element is provided which exhibits good sensitivity while having appropriate strength. The detector element forms part of a liquid-condition detection sensor in which breakage of the detection element is combated and which can accurately detect the condition of liquid. The detection element includes first and second ceramic insulating layers formed by simultaneous firing and a heat-generating resistor sealed in a liquid tight manner between the insulating layers. The resistor has a resistance varying with temperature, and is, in use, immersed in liquid. The first insulating layer is thinner than the second layer. A detection section of the liquid-condition detection sensor detects the condition of a liquid (e.g., the concentration of a particular component in the liquid) on the basis of an output signal from the heat-generating resistor.

### TECHNICAL FIELD

The present invention relates to a liquid-condition detection element and a liquid-condition detection sensor using the same.

### BACKGROUND ART

Some exhaust gas purification systems for reducing nitrogen oxides (NOₓ) emitted from a diesel-powered automobile use an NOₓ selective catalytic reduction (SCR) system. The SCR system uses a urea aqueous solution as a reducing agent. Using a urea aqueous solution having a urea concentration of 32.5 wt % is known to be effective for efficiently carrying out the reducing reaction. However, the urea aqueous solution contained in an aqueous urea tank mounted in a diesel-powered automobile may vary in urea concentration because of variations over time or for other reasons. Also, a different solution (e.g., light oil), water, or the like may be erroneously mixed in the aqueous urea tank. In view of these problems, in order to control the condition of liquid (i.e., the urea concentration in urea aqueous solution) in the aqueous urea tank, a liquid-condition detection sensor (i.e., a urea-concentration-determining device) has been proposed (reference is made to, for example, Japanese Patent Application Laid-Open (kokai) No. 2005-84026).

The urea-concentration-determining device described in the above-mentioned reference includes an indirectly heated concentration-detection section having an element (i.e., a thin-film chip) in a laminated structure in which a substrate, a temperature-sensing element, an insulating layer, a heat-generating element, and a protective layer are laminated together in this sequence. In this urea-concentration-determining device, the heat-generating element is energized for a predetermined period of time. On the basis of the variation in temperature of the heat-generating element before and after energization of the heat-generating element as measured by the temperature-sensing element, the concentration of urea is determined. Specifically, since a difference in the concentration of urea in a urea aqueous solution causes a difference in the thermal capacity of the urea aqueous solution, a difference in the concentration of urea causes a difference in temperature variation of the heat-generating element. Thus, the concentration of urea is determined through detection of the variation in temperature of the heat-generating element.

### SUMMARY OF THE INVENTION

A disadvantage of the urea-concentration-determining device described above is that, in order to prevent entry of a urea aqueous solution into the element (i.e., the thin-film chip), the element (the thin-film chip) is molded with resin. In this urea-concentration-determining device, since the thermal conductivity of resin is low, conduction of heat to the urea aqueous solution is difficult. Thus, molding the element (the thin-film chip) with resin causes difficulty in raising the temperature of the urea aqueous solution. Therefore, the difference in temperature variation of the temperature-sensing element derived from a difference in the concentration of urea in the urea aqueous solution is small and difficult to detect. In other words, the urea-concentration-determining device does not exhibit good sensitivity in detecting changes in the temperature of the element (the thin-film chip) and thus fails to provide an accurate determination of the concentration of urea.

One important aspect of the present invention concerns overcoming the problems of the prior art device. To this end, there is provided a liquid-condition detection sensor having a liquid-condition detection element (hereinafter sometimes referred to merely as “element”) configured such that a heat-generating resistor is sealed in a ceramic substrate formed in a laminated structure comprised of a first ceramic insulating layer and a second ceramic insulating layer (refer to Japanese Patent Application No. 2005-200808). Sealing the heat-generating resistor in the ceramic laminate eliminates the risk of entry of liquid into the element. Thus, the element can be directly immersed in the liquid. Therefore, as compared with an element molded with resin as in the device described above, sensitivity is enhanced.

In recent years, the demand has been increasing for liquid-condition detection elements having higher sensitivity. The thinner the ceramic insulating layers which cover the heat-generating resistor, the greater the reduction in the heat removed by the ceramic insulating layers, i.e., the more heat that will be conducted to liquid, thereby enhancing the sensitivity of the liquid-condition detection element. However, as the thickness of the ceramic insulating layers is reduced, the mechanical strength (also referred to hereinafter as the “strength”) of the element is decreased. Thus, a liquid-condition detection sensor which uses such an element may suffer with respect to reliability.

More particularly, in the case where the ceramic substrate used to form the element is in direct contact with the liquid when detecting the condition of the liquid, if the element is subjected to a temperature at which the liquid freezes, repetitively energizing and de-energizing of the heat-generating resistor is accompanied by repeated melting and freezing of the liquid around the element. The associated great change in the volume of the liquid (solid) results in a large force being exerted on the element. In order to prevent breakage of the element, the strength of the element cannot be excessively lowered, i.e., there are limits on how much the strength of the element can be reduced.

One aspect of the present invention concerns the appreciation of the foregoing problems, and one object of the invention is to provide a liquid-condition detection element which exhibits good sensitivity while having adequate or appropriate strength, and to provide a liquid-condition detection sensor in which breakage of the liquid-condition detection element is essentially suppressed and which provides accurate detection of the condition of the liquid.

According to one aspect of the invention there is provided a liquid-condition detection element comprising a heat-generating resistor sealed, in a liquid tight manner, between a first ceramic insulating layer and a second ceramic insulating layer and having a resistance varying with temperature thereof. When the liquid-condition detection element is immersed in liquid and the heat-generating resistor is energized, the heat-generating resistor produces an output signal relating to a condition of the liquid. Preferably, the first ceramic insulating layer has a thickness smaller than that of the second ceramic insulating layer.

The heat generating resistor of the liquid-condition detection element has a resistance that varies with the temperature thereof. Accordingly, through energization of the heat-generating resistor, the liquid-condition detection element can produce an output signal corresponding to the resistance of the heat-generating resistor, in accordance with the condition of the liquid. On the basis of this signal, the condition of the liquid can be determined.

Also, in the liquid-condition detection element of this aspect of the present invention, the first ceramic insulating layer and the second ceramic insulating layer used in providing liquid-tight sealing of the heat-generating resistor therebetween differ in thickness; specifically, as indicated above, the first ceramic insulating layer is thinner than the second ceramic insulating layer. For a given overall thickness of the detection element, as compared with, for example, a liquid-condition detection element wherein the first and second ceramic insulating layers have the same thickness, the liquid-condition detection element with the thinner first layer exhibits better sensitivity while having similar strength. It is theorized that this occurs for the following reason. The thinner the ceramic insulating layers which cover the heat-generating resistor, the greater the reduction in heat that is removed by the ceramic insulating layers, i.e., the more likely that the heat is conducted to the liquid rather than to the insulating layers. For a given overall thickness of the detection element, as compared with a liquid-condition detection element wherein first and second ceramic insulating layers have the same thickness, the liquid-condition detection element of the present invention allows heat to be more readily conducted to liquid through the first ceramic insulating layer. Accordingly, the temperature of the heat-generating resistor is more susceptible to, i.e., provides a better indication of, the condition of liquid (e.g., the concentration of a particular component in the liquid). This is because a difference in the condition of the liquid (e.g., the concentration of a particular component in the liquid) causes a significant difference in conducting heat to the liquid.

Accordingly, in the liquid-condition detection element of the present invention, the difference in the resistance of the heat-generating resistor derived from the difference in the condition of liquid is relatively large; thus, the difference in an output signal output from the heat-generating resistor increases. In other words, the detection sensitivity is improved. Thus, for a given overall thickness of the detection element, as compared with a liquid-condition detection element in which the first ceramic insulating layer and the second ceramic insulating layer have the same thickness, the liquid-condition detection element of the present invention can exhibit better sensitivity while having similar strength.

Therefore, the liquid-condition detection element of the present invention exhibits good sensitivity while having an adequate or appropriate strength.

Preferably, in the above-mentioned liquid-condition detection element, the heat-generating resistor is of a meandering shape comprising a plurality of, and preferably a large number of, parallel, linear portions extending in parallel with one another and a plurality of, and preferably a large number of, connection portions each connecting adjacent parallel, linear portions, and the parallel, linear portions are arranged at intervals smaller than the thickness of the first ceramic insulating layer.

By arranging the parallel, linear portions of the heat-generating resistor at intervals smaller than the distance between the surface of the first ceramic insulating layer and the parallel, linear portions (i.e., the thickness of the first ceramic insulating layer), any variation in temperature distribution on the surface of the first ceramic insulating layer can be made small, thereby reducing any corresponding unevenness in the heating of the liquid. By virtue of this feature, combined with the effect yielded by making the first ceramic insulating layer thinner than the second ceramic insulating layer, the condition of the liquid can be detected with higher accuracy.

It is noted that no particular limitation is imposed on the shape of each of the parallel, linear portions so long as a large number of linear portions extend in parallel with one another. For example, either straight-line portions or curved-line portions may be provided so long as they extend in parallel with one another.

Preferably, in the above-mentioned liquid-condition detection element, an outer surface of the ceramic substrate includes a contact zone which comes into contact with the liquid.

The liquid-condition detection element of the present invention exhibits adequate or appropriate strength as mentioned above. Thus, even when the element is subjected to a temperature condition under which the liquid freezes, and the heat-generating resistor is repetitively energized and de-energized, breakage of the element can be effectively prevented. Direct contact of the ceramic substrate with liquid accelerates conduction of heat to the liquid through the thin first ceramic insulating layer, thereby maximizing the effect of improving sensitivity.

Preferably, in either of the above-mentioned liquid-condition detection elements, the first ceramic insulating layer and the second ceramic insulating layer are of the same material. This feature is important because if the first ceramic insulating layer and the second ceramic insulating layer are made of the same material, each will have the same coefficient of thermal expansion. Accordingly, the first ceramic insulating layer and the second ceramic insulating layer expand or contract with temperature to a similar degree, thereby preventing strain and breakage of the liquid-condition detection element, which could otherwise arise from a difference in expansion/contraction between the first and second ceramic insulating layers. Therefore, breakage of the liquid-condition detection element of the present invention is suppressed to a greater extent.

Preferably, in any one of the above-mentioned liquid-condition detection elements, the first ceramic insulating layer and the second ceramic insulating layer are formed by simultaneous firing. This is important because forming the first ceramic insulating layer and the second insulating layer through simultaneous firing enhances bonding strength between the first and second ceramic insulating layers. This enhances sealing the heat-generating resistor in the ceramic substrate, thereby enhancing the reliability of the liquid-condition detection element.

Preferably, in any one of the above-mentioned liquid-condition detection elements, connection conductors electrically communicating with the heat-generating resistor extend through the first ceramic insulating layer across thickness thereof, and the output signal is output through the connection conductors. This feature has the advantage that in formation of the connection conductor to electrically communicate with the heat-generating resistor, along the direction of thickness of the ceramic substrate, the connection conductor is formed in such a manner as to extend through the thin first ceramic insulating layer across the thickness thereof, thereby reducing the amount of material used to form the connection conductor. This lowers the cost of the liquid-condition detection element.

Preferably, in any one or all of the above-mentioned liquid-condition detection elements, the liquid is a urea aqueous solution.

The liquid-condition detection element of the present invention is immersed in a urea aqueous solution. The urea aqueous solution is contained as an NOₓ-reducing agent in, for example, a liquid container of a diesel-powered vehicle. In a low-temperature environment, such as that encountered during winter, the urea aqueous solution may freeze. In such a low-temperature environment, repetitively energizing and de-energizing the heat-generating resistor of the liquid-condition detection element immersed in the urea aqueous solution causes repeated melting and freezing of the urea aqueous solution around the liquid-condition detection element. An associated large change in volume of the urea aqueous solution may exert a large force on the liquid-condition detection element.

As mentioned previously, the liquid-condition detection element of the present invention has appropriate strength and exhibits good sensitivity. Thus, in the course of use of the liquid-condition detection element of the present invention in a low-temperature environment, the element is free from breakage, which could otherwise result from a change in conditions (freezing and melting) of the urea aqueous solution, and can detect the condition of the urea aqueous solution with good accuracy.

In accordance with a further aspect of the invention, there is provided a liquid-condition detection sensor comprising a liquid-condition detection element and a detection section. The liquid-condition detection element comprises a ceramic substrate comprising a laminated structure comprising a first ceramic insulating layer and a second ceramic insulating layer, and a heat-generating resistor sealed in a liquid-tight manner between the first ceramic insulating layer and the second ceramic insulating layer and having a resistance varying with the temperature thereof. The liquid-condition detection element is immersed in liquid. The detection section detects a condition of the liquid on the basis of an output signal which the energized heat-generating resistor outputs or produces in accordance with resistance thereof. The first ceramic insulating layer is smaller in thickness than the second ceramic insulating layer.

The liquid-condition detection sensor includes the liquid-condition detection element which, in turn, includes the heat-generating resistor the resistance of which varies with the temperature thereof and which is immersed in liquid. Thus, through energization of the heat-generating resistor, the liquid-condition detection element produces an output signal corresponding to the resistance of the heat-generating resistor. On the basis of this signal, the liquid-condition detection sensor can determine the condition of the liquid.

Also, in the liquid-condition detection element of the liquid-condition detection sensor of the present invention, the first ceramic insulating layer and the second ceramic insulating layer used for providing liquid-tight sealing of the heat-generating resistor therebetween differ in thickness; specifically, the first ceramic insulating layer is thinner than the second ceramic insulating layer. For a given overall thickness of the detector element, as compared with, for example, a liquid-condition detection element whose first and second ceramic insulating layers have the same thickness, the liquid-condition detection element of the liquid-condition detection sensor of the present invention exhibits better sensitivity while having similar strength. The reason for this was discussed above. Therefore, the liquid-condition detection element of the liquid-condition detection sensor of the present invention exhibits good sensitivity while having appropriate strength.

Thus, in the liquid-condition detection sensor of the present invention, the liquid-condition detection element is unlikely to break. Also, the liquid-condition detection sensor can detect the condition of liquid with good accuracy.

Examples of an “output signal” which the energized heat-generating resistor outputs in accordance with resistance thereof include a “voltage” which arises from application of constant current to the heat-generating resistor, and a “current” which arises from application of constant voltage to the heat-generating resistor.

Preferably, in the above-mentioned liquid-condition detection sensor, the heat-generating resistor is of a meandering shape comprising a large number of parallel, linear portions extending in parallel with one another and a large number of connection portions each connecting adjacent parallel, linear portions, and the parallel, linear portions are arranged at intervals smaller than the thickness of the first ceramic insulating layer.

By arranging the parallel, linear portions of the heat-generating resistor at intervals smaller than the distance between the surface of the first ceramic insulating layer and the parallel, linear portions (i.e., the thickness of the first ceramic insulating layer), any variation in temperature distribution on the surface of the first ceramic insulating layer can be made small, thereby reducing any unevenness in the heating of the liquid. By virtue of this feature, combined with the effect yielded by making the first ceramic insulating layer thinner than the second ceramic insulating layer, the condition of liquid can be detected with higher accuracy.

Preferably, in the above-mentioned liquid-condition detection sensor, an outer surface of the ceramic substrate includes a contact zone which comes into contact with the liquid.

In the liquid-condition detection sensor of the present invention, the liquid-condition detection element exhibits appropriate strength as mentioned above. Thus, even when the element is subjected to a temperature condition under which the liquid freezes, and the heat-generating resistor is repetitively energized and de-energized, breakage of the element can be effectively prevented. Direct contact of the ceramic substrate with the liquid accelerates conduction of heat to the liquid through the thin first ceramic insulating layer, thereby maximizing the effect of improving sensitivity.

Preferably, in either of the above-mentioned liquid-condition detection sensors, the liquid-condition detection element is such that the first ceramic insulating layer and the second ceramic insulating layer are of the same material.

The first ceramic insulating layer and the second ceramic insulating layer of the same material preferably have the same coefficient of thermal expansion. Accordingly, the first ceramic insulating layer and the second ceramic insulating layer expand or contract with temperature to a similar degree, thereby preventing strain and breakage of the liquid-condition detection element, which could otherwise arise from a difference in expansion/contraction between the first and second ceramic insulating layers. Therefore, breakage of the liquid-condition detection element of the liquid-condition detection sensor of the present invention is suppressed to a greater extent.

Preferably, in any one of the above-mentioned liquid-condition detection sensors, the liquid-condition detection element is such that the first ceramic insulating layer and the second ceramic insulating layer are formed by simultaneous firing.

As discussed above, forming the first ceramic insulating layer and the second insulating layer through simultaneous firing enhances the bonding strength between the first and second ceramic insulating layers. This enhances sealing the heat-generating resistor in the ceramic substrate, thereby enhancing the reliability of the liquid-condition detection element and, in turn, the reliability of the liquid-condition detection sensor.

Preferably, in any one of the above-mentioned liquid-condition detection sensors, the liquid-condition detection element is such that connection conductors electrically communicating with the heat-generating resistor extend through the first ceramic insulating layer across thickness thereof and such that the output signal is output through the connection conductors.

As mentioned previously, in formation of the connection conductor to electrically communicate with the heat-generating resistor, along the direction of thickness of the ceramic substrate, the connection conductor is formed in such a manner as to extend through the thin first ceramic insulating layer across the thickness thereof, thereby reducing the amount of material used to form the connection conductor. This lowers the cost of the liquid-condition detection element and, in turn, the cost of the liquid-condition detection sensor.

Preferably, in any one of the above-mentioned liquid-condition detection sensors, the detection section energizes the heat-generating resistor for a predetermined period of time, obtains a first correspondence value and a second correspondence value corresponding to respective resistances of the heat-generating resistor at different times within the predetermined period of time, and determines at least the concentration of a particular component in the liquid on the basis of the first correspondence value and the second correspondence value.

Through employment of the above configuration for the detection section, the degree of temperature rise of the heat-generating resistor can be precisely captured or determined, whereby the concentration of a particular component in liquid can be accurately and stably detected.

It is noted that the aforementioned “first correspondence value” and “second correspondence value” corresponding to respective resistances of the heat-generating resistor may be values having the same units, and examples thereof include voltage, current, and a value as reduced to temperature. The concentration of a particular component in the liquid can be detected by use of, for example, the difference between the first and second correspondence values or the ratio between the first and second correspondence values.

Preferably, the above-mentioned liquid-condition detection sensor further comprises a level detector having a first electrode and a second electrode, and the first and second electrodes form a capacitor the capacitance of which varies with the level of the liquid, and the liquid-condition detection element is united in or integrated with an insulated condition with the level detector.

In the liquid-condition detection sensor of the present invention, the capacitance-based level detector, i.e., the level detector for detecting the level of liquid in accordance with the variation in capacitance, with the liquid level, and the liquid-condition detection element are united or integrated in an insulated manner with each other. By uniting the capacitance-based level detector, the level detection accuracy of which is relatively high, with the liquid-condition detection element, a single sensor can detect both the level of liquid and the concentration of a particular component in the liquid with good accuracy.

Preferably, in any one of the above-mentioned liquid-condition detection sensors, the liquid is a urea aqueous solution.

As discussed above in connection with the liquid-condition detection element, the liquid-condition detection sensor of the present invention is adapted to detect the condition of a urea aqueous solution and the urea aqueous solution is typically contained as an NOₓ-reducing agent in, for example, a liquid container of a diesel-powered vehicle. In a low-temperature environment, such as that encountered during winter, the urea aqueous solution may freeze. In such a case, repetitively energizing and de-energizing the heat-generating resistor of the liquid-condition detection element causes repeated melting and freezing of the urea aqueous solution around the liquid-condition detection element. An associated large change in volume of the urea aqueous solution may exert a large force on the liquid-condition detection element.

In the liquid-condition detection sensor of the present invention, as mentioned previously, the liquid-condition detection element has appropriate strength and exhibits good sensitivity. Thus, in a low-temperature environment, the liquid-condition detection sensor of the present invention is not subject to breakage of the liquid-condition detection element, which could otherwise result from a change in conditions (freezing and melting) of the urea aqueous solution, and can detect the condition of the urea aqueous solution with good accuracy.

Further features and advantages of the present invention will be set forth in, or apparent from, the detailed description of preferred embodiments thereof which follows.

### BRIEF DESCRIPTION OF THE DRAWINGS

FIG. 1 is a front elevational view, partially in cross section, of a liquid-condition detection sensor according to one embodiment of the invention;

FIGS. 2 is a cross-sectional view of a liquid-condition detection element according to the embodiment of FIG. 1;

FIG. 3 is a cross-sectional view used in describing the interior of the liquid-condition detection element;

FIG. 4 is a block diagram showing the electrical configuration of the liquid-condition detection sensor of FIG. 1;

FIG. 5 is a graph showing an example of the variation in voltage V of a heat-generating resistor at the time of energization; and

FIG. 6 is a graph showing the relation between the thickness of ceramic insulating layers and sensitivity (DV differential/V1).

### DESCRIPTION OF THE PREFERRED EMBODIMENTS

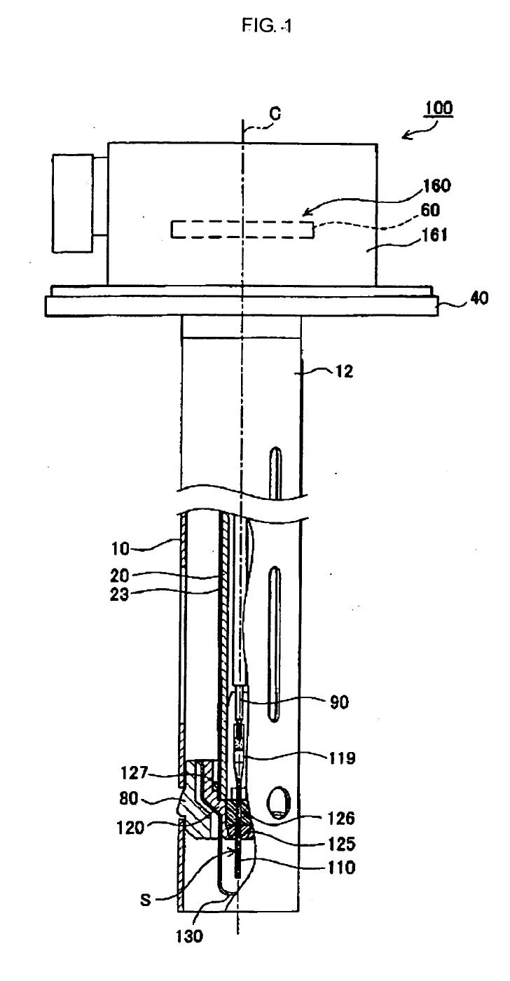

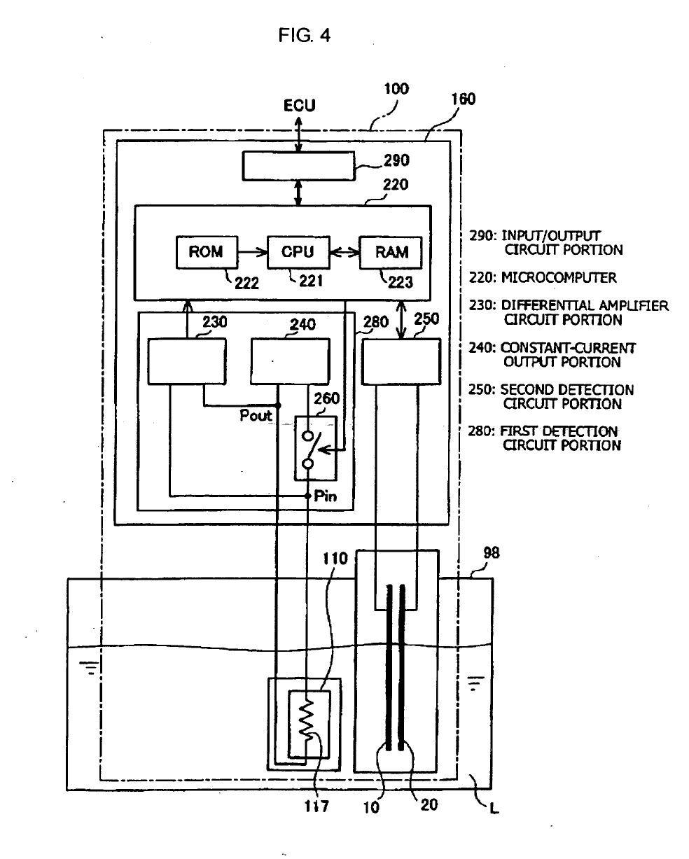

An embodiment of the present invention will be described with reference to the drawings. FIG. 1 is a partial cross-sectional view of a liquid-condition detection sensor 100 according to one preferred embodiment of the present invention. As shown in FIG. 1, the liquid-condition detection sensor 100 includes a liquid-condition detection element 110, an outer, tubular electrode 10, an inner, tubular electrode 20, a detection section 160, and a mounting section 40. In the liquid-condition detection sensor 100, as viewed along a longitudinal axis C, the side facing toward the liquid-condition detection element 110 is a front end side, and the side facing toward the detection section 160 is a rear end side. As shown in FIG. 4, in the liquid-condition detection sensor 100 of this embodiment, a front end portion thereof is immersed in a urea aqueous solution L contained in an aqueous-urea tank 98, whereby the condition of the urea aqueous solution L can be detected.

The mounting section 40 is made of metal and has bolt through-holes (not shown) formed therein for allowing corresponding bolts to be inserted therethrough. The liquid-condition detection sensor 100 can be mounted to the aqueous-urea tank 98 (see FIG. 4) by use of mounting bolts to be inserted through the bolt through-holes of the mounting section 40.

The outer, tubular electrode 10 is made of metal, is of a cylindrical shape, and extends rearwardly from the front end side of the liquid-condition detection sensor 100 with its axis coinciding with the axis C. The outer, tubular electrode 10 is welded at its rear end portion 12 to the mounting section 40. The mounting section 40 is connected to a wiring board 60, which is a component of the detection section 160, in such a manner as to have the same potential as that of a wiring section (not shown) of ground potential, i.e., connected to ground. Accordingly, the outer, tubular electrode 10 welded to the mounting section 40 is at ground potential.

The inner, tubular electrode 20 is made of metal, is of a cylindrical shape smaller in diameter than the outer, tubular electrode 10, and is disposed such that its axis coincides with the axis C. The inner, tubular electrode 20 extends rearwardly, within the outer, tubular electrode 10, from the front end side of the liquid-condition detection sensor 100. Although this is not illustrated, the inner, tubular electrode 20 is fixedly attached, at its rear end portion, to the mounting section 40 via an insulating member. The inner, tubular electrode 20 is electrically connected to the wiring board 60, which is a component of the detection section 160. An AC voltage is applied to the inner, tubular electrode 20. The outer surface of the inner, tubular electrode 20 in contact with the urea aqueous solution L is coated with an insulating film 23 of fluorine-containing resin.

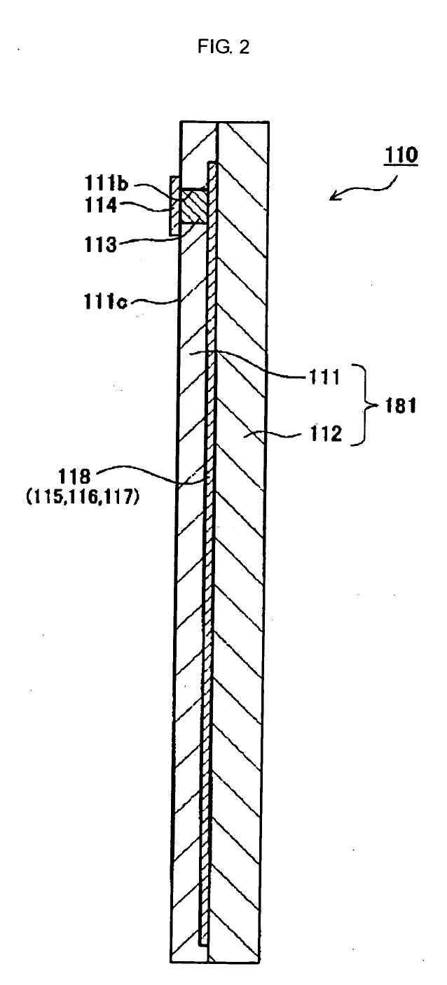

As shown in FIG. 2, the liquid-condition detection element 110 includes a first ceramic insulating layer 111, a second ceramic insulating layer 112, and a conductor layer 118 positioned therebetween. More specifically, the liquid-condition detection element 110 is formed by simultaneous firing so that a liquid tight seal is provided between the conductor layer 118 sealed between the first ceramic insulating layer 111 and the second ceramic insulating layer 112. Accordingly, even when the liquid-condition detection element 110 is directly immersed in the urea aqueous solution L, the conductor layer 118 is free from any short circuiting thereof, which could otherwise result from entry of the urea aqueous solution L into the liquid-condition detection element 110. The liquid-condition detection element 110 is directly immersed in the urea aqueous solution L, and the outer surface of a front end portion thereof (ceramic substrate 181) serves as a contact zone S (see FIG. 1) which comes into contact with the urea aqueous solution L. Therefore, as compared with an element molded with resin, the liquid-condition detection element 110 exhibits better sensitivity.

In this embodiment, the first ceramic insulating layer 111 and the second ceramic insulating layer 112 are both made of alumina and assume the form of, i.e., have the shape of, a rectangular plate. The first and second ceramic insulating layers 111 and 112 are stacked into a single ceramic substrate 181. However, in this embodiment, as shown in FIG. 2, the first ceramic insulating layer 111 is smaller in thickness than the second ceramic insulating layer 112. Specifically, a preferred thickness of the first ceramic insulating layer 111 is, for example, 0.27 mm and a preferred thickness of the second ceramic insulating layer 112 is, for example, 0.39 mm.

When the strength of the element is to be enhanced, preferably only the thickness of the second ceramic insulating layer 112 is increased while the thickness of the first ceramic insulating layer 111 is held fixed or unchanged. While the strength of the element is enhanced by increasing the overall thickness of the element, the conductivity of heat to the urea aqueous solution through the first ceramic insulating layer 111 will be substantially unchanged if the thickness of the first ceramic insulating layer 111 is unchanged, thereby suppressing a drop in sensitivity of the element. Specifically, by employing, for example, a thickness of the first ceramic insulating layer 111 of 0.27 mm and a thickness of the second ceramic insulating layer 111 of 0.59 mm or 0.80 mm, the strength of the element can be enhanced while any drop in the sensitivity of the element is suppressed.

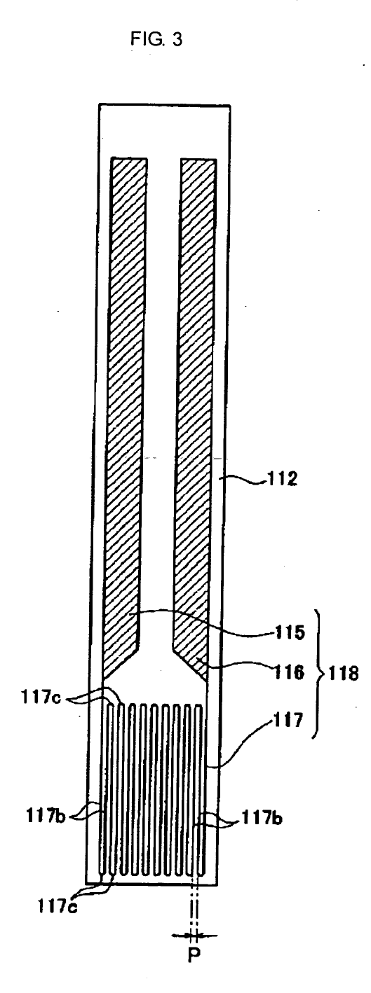

The conductor layer 118 preferably contains Pt as a main component and includes, as shown in FIG. 3, a first lead portion 115, a second lead portion 116, and a heat-generating resistor 117 connected thereto. The heat-generating resistor 117 has a large number of parallel, linear portions 117b extending straight, in parallel with one another, in the longitudinal direction of the second ceramic insulating layer 112 (in the vertical direction as viewed in FIG. 3) and arcuate connection portions 117c each connecting the adjacent parallel, linear portions 117b in such a manner as to change directions, as illustrated. The heat-generating resistor 117, as a whole has the form of a meandering wire having a cross-sectional area smaller than that of the first lead portion 115 and the second lead portion 116. Accordingly, when the conductor layer 118 is energized, heat is generated mainly in the heat-generating resistor 117. The heat-generating resistor 117 varies in resistance with the temperature thereof.

Also, according to this embodiment, in the heat-generating resistor 117, a large number of the parallel, linear portions 117b are arranged at intervals P of 0.15 mm in the lateral direction (the left to right direction as viewed in FIG. 3) of the second ceramic insulating layer 112. More specifically, the intervals P between the parallel, linear portions 117b are made smaller than the thickness (0.27 mm) of the first ceramic insulating layer 111, which is thinner than the second ceramic insulating layer 112.

By making the intervals P between the parallel, linear portions 117b, which are major heat-generating portions of the heat-generating resistor 117, smaller than the distance between the parallel, linear portions 117b and a surface 111c of the first ceramic insulating layer 111 in contact with the urea aqueous solution L (i.e. the thickness of the first ceramic insulating layer 111), any variation in the temperature distribution on the surface 111c of the first ceramic insulating layer 111 is made small, thereby reducing any unevenness in the heating of the urea aqueous solution L located around the liquid-condition detection element 110. Thus, the condition of the urea aqueous solution L can be detected with good accuracy.

Furthermore, as shown in FIG. 2, the first ceramic insulating layer 111 has two via holes 111b extending therethrough across the thickness thereof (in the left to right direction in FIG. 2) at positions corresponding to the conductor layer 118 (specifically, the first lead portion 115 and the second lead portion 116). The via holes 111b are filled with respective via conductors 113. Square connection pads 114 electrically communicating with the respective via conductors 113 are formed on the surface 111c of the first ceramic insulating layer 111.

Connectors 119 are connected to the respective connection pads 114 (see FIG. 1). As shown in FIG. 1, the connectors 119 and the detection section 160 (wiring board 60) are electrically connected together by lead wires 90 extending through the inner, tubular electrode 20. Thus, the heat-generating resistor 117 of the liquid-condition detection element 110 is electrically connected to the detection section 160 (wiring board 60). The via conductors 113 electrically communicating with the heat-generating resistor 117 correspond to the “connection conductor” discussed herein. It will be understood that the connection conductor is not limited to a via conductor filled into the via hole 111b, but may also take the form of conductor formed on the wall of the via hole 111b. Instead of the first ceramic insulating layer 111, the second ceramic insulating layer 112 may have the via conductors 113, which are provided therein in such a manner as to extend therethrough across the thickness thereof. However, providing this in the first ceramic insulating layer 111, which is thinner than the second ceramic insulating layer 112, can reduce material used to form the via conductors 113, thereby lowering the cost of the liquid-condition detection element 110.

While extending through an insulating tubular holder 120, which is attached to the inner, tubular electrode 20 via an annular seal member 127, the thus-configured liquid-condition detection element 110 is held by the holder 120 by means of fixing members 125 and 125, which are preferably formed of an insulating adhesive which fills the holder 120. A portion of the liquid-condition detection element 110 at which the heat-generating resistor 117 is located (i.e., outer surface of the ceramic substrate 181) projects forwardly (downwardly as viewed in FIG. 1) from the holder 120 so as to be immersed in the urea aqueous solution.

A tubular rubber bush 80 fixed to the outer, tubular electrode 10 fixes the holder 120 holding the liquid-condition detection element 110, to the inner, tubular electrode 20 in such a manner that the liquid-condition detection element 110 does not move along the axis C. The fixing members 125 and 126 which fill the holder 120 prevent entry of the urea aqueous solution L into the interior of the inner, tubular electrode 20. A protector 130, which surrounds and protects the liquid-condition detection element 110, is attached to the holder 120. The protector 130 has a plurality of through-holes for allowing flow of the urea aqueous solution L between the interior and exterior thereof. In this manner, in this embodiment, the insulating holder 120 is attached to a front end portion of the inner, tubular electrode 20, and the holder 120 is fixed to the outer, tubular electrode 10 via the rubber bush 80. Thus, the liquid-condition detection element 110, which is held in an insulated condition by the holder 120, is united in an insulated condition with a level detector (specifically, the inner, tubular electrode 20, which is a component of the level detector) which will be described later.

As shown in FIG. 1, the detection section 160 is implemented as the wiring board 60 on which a CPU and the like are mounted, and is disposed within a protection cover 161. Specifically, as shown in FIG. 4, the detection section 160 includes a microcomputer 220, a first detection circuit portion 280, a second detection circuit portion 250, and an input/output circuit portion 290.

The microcomputer 220 includes a CPU 221, a ROM 222, and a RAM 223 and performs various kinds of control. The input/output circuit portion 290 controls communication protocol for transmission of signals between the microcomputer 220 and an ECU (engine control unit).

Under an instruction from the microcomputer 220, the second detection circuit portion 250 applies a predetermined AC voltage between the outer, tubular electrode 10 and the inner, tubular electrode 20. The second detection circuit portion 250 converts the current flowing at this time, into a corresponding voltage, and outputs an associated voltage signal to the microcomputer 220. Since the capacitance between the outer, tubular electrode 10 and the inner, tubular electrode 20 varies with the amount of the urea aqueous solution L present between the outer, tubular electrode 10 and the inner, tubular electrode 20, the microcomputer 220 can determine the level of the urea aqueous solution L on the basis of the output voltage signal. As will be understood from the preceding description, in this embodiment, the outer, tubular electrode 10 serving as a first electrode and the inner, tubular electrode 20 coated with the insulating film 23 and serving as a second electrode, are disposed in opposition to each other, thereby forming a level detector, which is implemented as a capacitor the capacitance of which varies with the level of liquid.

The first detection circuit portion 280 includes a differential amplifier circuit portion 230, a constant-current output portion 240, and a switch 260. Under an instruction from the microcomputer 220, the first detection circuit portion 280 applies constant current to the liquid-condition detection element 110 and outputs a voltage signal corresponding to resistance of the heat-generating resistor 117 to the microcomputer 220.

Specifically, the constant-current output portion 240 is electrically connected to the heat-generating resistor 117 and outputs constant current. The switch 260 is connected in an energization line extending between the constant-current output portion 240 and the heat-generating resistor 117. Under an instruction from the microcomputer 220, the switch 260 switches ON/OFF the application of electricity to the heat-generating resistor 117 from the constant-current output portion 240. The differential amplifier circuit portion 230 outputs, as a detected voltage, the difference between potential Pin at the input end of the heat-generating resistor 117 and potential Pout at the output end of the heat-generating resistor 117 to the microcomputer 220. On the basis of this detected voltage, the microcomputer 220 can, for example, calculate the concentration of urea in the urea aqueous solution L to thereby determine whether or not the concentration of urea is proper, and can calculate the temperature of the urea aqueous solution L.

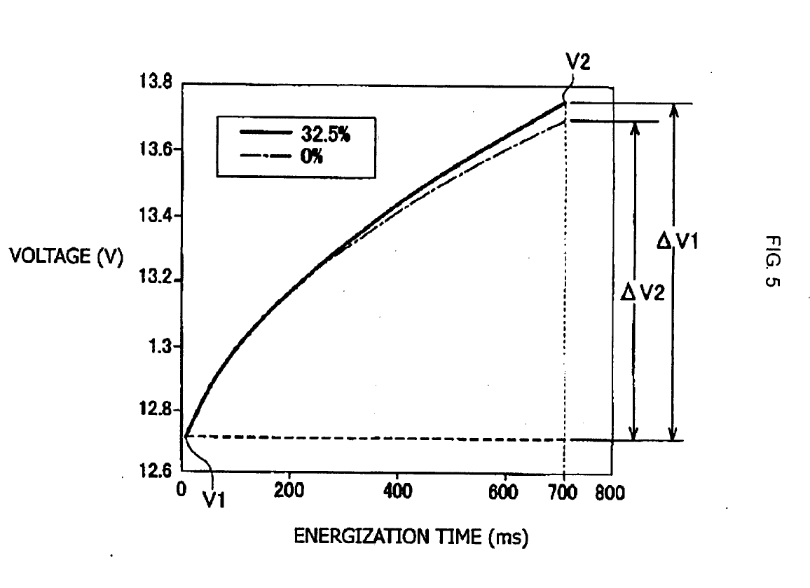

For example, when the concentration of urea in the urea aqueous solution L is 32.5 wt %, as illustrated by the solid line in FIG. 5, the voltage of the heat-generating resistor 117 varies with energization time. This operation will be described with reference to an example. First, the constant-current output portion 240 applies constant current to the heat-generating resistor 117. Substantially immediately after energization of the heat-generating resistor 117 is started (specifically, 10 msec after start of energization of the heat-generating resistor 117), there is detected a voltage signal (first detected voltage V1) which is output in correspondence with the resistance of the heat-generating resistor 117 and serves as a first correspondence value. Next, after elapse of a predetermined energization time t1 (e.g., t1=700 msec) from start of energization, there is detected a voltage signal (second detected voltage V2) which is output in correspondence with resistance of the heat-generating resistor 117 and serves as a second correspondence value.

Next, a difference ΔV (in this example, ΔV1) between V2 and V1, i.e., V2−V1, is calculated. If this ΔV (in this example, ΔV1) is equal to or less than a threshold value Q (the maximum value among ΔV values which have previously been obtained for urea aqueous solutions L of various concentrations), it can be determined that the urea aqueous solution L is contained in the aqueous-urea tank 98. Furthermore, by calculating the concentration of urea in the urea aqueous solution in accordance with a predetermined operational expression, it can be determined whether or not the concentration of urea is appropriate. In the example shown in FIG. 5, the urea concentration is calculated as 32.5 wt %; thus, the concentration of urea is judged appropriate.

The operation described above is based on the following principle. The difference in the concentration of urea in the urea aqueous solution L causes a difference in the thermal conductivity of the urea aqueous solution L. Thus, when the urea aqueous solution L is heated by the heat-generating resistor 117, a difference in the concentration of urea causes a difference in the rate of temperature rise of the urea aqueous solution L. Accordingly, the rate of temperature rise of the urea aqueous solution L (i.e., the concentration of the urea aqueous solution L) influences the temperature rise of the heat-generating resistor 117 of the liquid-condition detection element 110 immersed in the urea aqueous solution L.

As mentioned previously, the heat-generating resistor 117 varies in resistance with the temperature thereof. Accordingly, after constant current is applied to the heat-generating resistor 117 for a predetermined period of time, a difference in the concentration of urea in the urea aqueous solution L, a difference in the type of liquid, or the like, causes a difference in resistance of the heat-generating resistor 117. Thus, when constant current is applied to the heat-generating resistor 117 for the predetermined energization time t1, a difference in the concentration of urea in the urea aqueous solution L or the like causes a difference in the difference ΔV between the first detected voltage V1 and the second detected voltage V2; i.e., V2−V1. Therefore, on the basis of ΔV, the concentration of urea in the urea aqueous solution L or the type of liquid can be determined.

The temperature of the heat-generating resistor 117 as measured immediately after start of energization is substantially equal to the temperature of the urea aqueous solution L present around the liquid-condition detection element 110 (heat-generating resistor 117). Thus, the resistance of the heat-generating resistor 117 as measured immediately after start of energization corresponds to the temperature of the urea aqueous solution L present around the liquid-condition detection element 110 (heat-generating resistor 117). Accordingly, by use of the first detected voltage V1, the temperature of the urea aqueous solution L can be determined.

Meanwhile, when ΔV is in excess of the threshold value Q, it indicates that an appropriate urea aqueous solution is not contained in the aqueous-urea tank 98. Specifically, when a liquid (specifically, light oil or the like) different from the urea aqueous solution L is contained in the aqueous-urea tank 98, ΔV can exceed the threshold value Q. Further, when the aqueous-urea tank 98 is empty, ΔV increases further.

Thus, by previously setting a threshold value R on the basis of ΔV obtained when the aqueous-urea tank 98 is empty, when a measured ΔV is in excess of the threshold value Q and is further in excess of the threshold valve R, the aqueous-urea tank 98 can be judged empty. When a measured ΔV is between the threshold value Q and the threshold value R, it can be determined that the aqueous-urea tank 98 is not empty, but contains liquid (light oil or the like) of a lower thermal conductivity than an appropriate urea aqueous solution L. In this manner, even an abnormality in the aqueous-urea tank 98 can be detected. Of course, such detection of an abnormality is a kind of detection of condition of the urea aqueous solution L.

In the method of determining the concentration of urea in the urea aqueous solution L on the basis of ΔV derived from a variation in resistance of the heat-generating resistor 117, the greater the difference in resistance of the heat-generating resistor 117 derived from a difference in concentration of the urea aqueous solution L (i.e., the higher the sensitivity of the liquid-condition detection element), the more that the difference in ΔV increases; as a result, the concentration of urea in the urea aqueous solution L can be determined with good accuracy.

The thinner the first and second ceramic insulating layers 111 and 112 which cover the heat-generating resistor 117, the greater the reduction in heat removed by the ceramic insulating layers; i.e., the more likely heat is conducted to the urea aqueous solution L, thereby enhancing the sensitivity of the liquid-condition detection element 110. However, as the thickness of the first and second ceramic insulating layers 111 and 112 is reduced, the mechanical strength of the liquid-condition detection element 110 is lowered.

More particularly, since the liquid-condition detection element 110 of the present embodiment is immersed in the urea aqueous solution L, under low-temperature conditions wherein the urea aqueous solution L freezes, repetitively energizing and de-energizing the heat-generating resistor is accompanied by repeated melting and freezing of the urea aqueous solution L. An associated great change in volume of the urea aqueous solution L exerts a great force on the element 110 (in other words, the contact zone S of the ceramic substrate 181 of the element 110 in contact with the urea aqueous solution L). Thus, a predetermined strength must be maintained while sensitivity is enhanced.

Thus, in an examination of sensitivity and strength, six kinds of liquid-condition detection elements (Samples 1to 6) having ceramic insulating layers of different thicknesses were prepared. Samples 1 to 6 employed similar heat-generating resistors 117.

Samples 1 to 3 were prepared as samples of the liquid-condition detection element 110 according to the embodiment described above.

In Sample 1, the first ceramic insulating layer 111 has a thickness of 0.27 mm, and the second ceramic insulating layer 112 has a thickness of 0.39 mm.

In Sample 2, the first ceramic insulating layer 111 has a thickness of 0.27 mm, and the second ceramic insulating layer 112 has a thickness of 0.59 mm.

In Sample 3, the first ceramic insulating layer 111 has a thickness of 0.27 mm, and the second ceramic insulating layer 112 has a thickness of 0.80 mm.

As mentioned above, according to the present embodiment, the first ceramic insulating layer 111 is thinner than the second ceramic insulating layer 112.

Samples 4 to 6 were prepared as samples of a liquid-condition detection element according to a comparative example.

In Sample 4, the first ceramic insulating layer 111 and the second ceramic insulating layer 112 both have a thickness of 0.27 mm.

In Sample 5, the first ceramic insulating layer 111 and the second ceramic insulating layer 112 both have a thickness of 0.39 mm.

In Sample 6, the first ceramic insulating layer 111 and the second ceramic insulating layer 112 both have a thickness of 0.59 mm.

Thus, as mentioned above, according to these comparative examples, the first ceramic insulating layer 111 and the second ceramic insulating layer 112 have the same thickness.

Samples 1 to 6 were examined for sensitivity. Specifically, liquid-condition detection sensors having respective Samples 1 to 6 were immersed in a urea aqueous solution L having a urea concentration of 32.5 wt % and were used to detect ΔV (as indicated above, the detected ΔV is referred to as ΔV1) by the aforementioned method (see FIG. 5). Similarly, the liquid-condition detection sensors were immersed in water having a urea concentration of 0 wt % and were used to detect ΔV (wherein the detected ΔV is referred to as ΔV2). The energization time t1 for the heat-generating resistor 117 was 700 msec for all Samples 1 to 6.

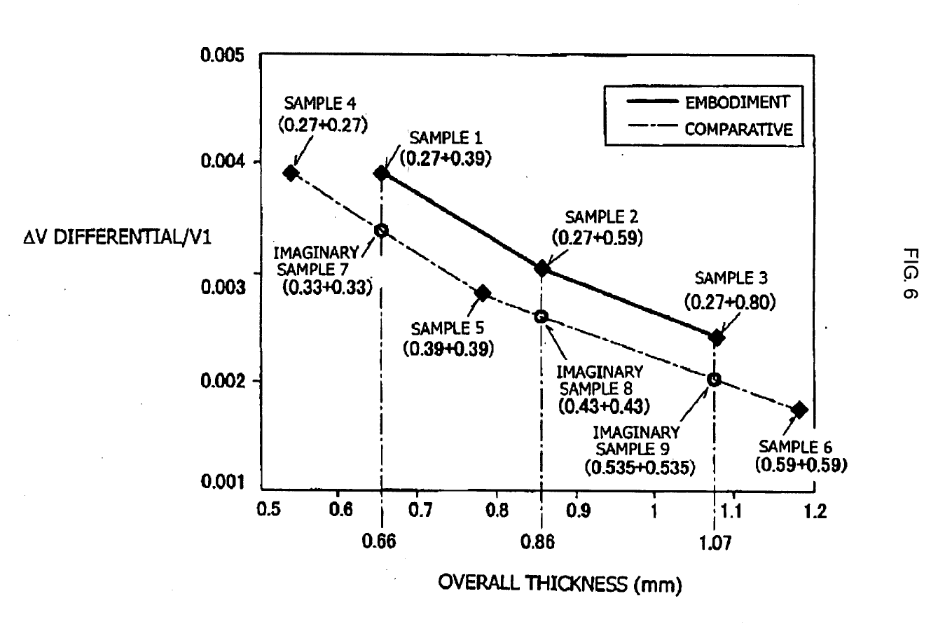

Next, ΔV differential between ΔV1 and ΔV2; i.e., ΔV1−ΔV2, was calculated. The greater the ΔV differential, the better the sensitivity. Since a manufacturing error in manufacture of the heat-generating resistors 117 may cause a fine or small difference in the natural resistance among the various heat-generating resistors 117 of Samples 1 to 6, a corrected value obtained by dividing the ΔV differential by the first detected voltage V1 (ΔV differential/V1) was used to compare sensitivity among the samples. FIG. 6 shows the results.

Sample 4 (0.27 mm+0.27 mm) according to the comparative example exhibits the highest ΔV differential/V1 value, indicating that Sample 4 has excellent sensitivity. However, Sample 4 has a problem with strength. Specifically, since the overall thickness is as thin as 0.54 mm, Sample 4 suffers the risk of breakage under such low-temperature conditions wherein the urea aqueous solution L freezes.

In contrast, Sample 1 (0.27 mm+0.39 mm) according to the present embodiment exhibited a ΔV differential/V1 value which was slightly smaller than, but was almost equivalent to, that of Sample 4, indicating that Sample 1 also has excellent sensitivity. Furthermore, since Sample 1 has a thickness of 0.66 mm which is 0.12 mm greater than the thickness of Sample 1, the strength thereof is enhanced. Therefore, Sample 1 is expected to be free from breakage even under such low-temperature conditions wherein the urea aqueous solution L freezes.

The sensitivity (ΔV differential/V1) will now be compared between the liquid-condition detection element 100 according to the present embodiment and a liquid-condition detection element according to the corresponding comparative example having the same overall thickness. Since the liquid-condition detection elements according to the present embodiment and the comparative example are formed by simultaneous firing, the liquid-condition detection elements, because they have the same thickness, can be said to have substantially the same strength.

First, Sample 1 (present embodiment) having an overall thickness of 0.66 mm and a sample of the comparative example having an overall thickness of 0.66 mm are compared. For this comparison, Imaginary Sample 7 (wherein the first and second insulating layers 111 and 112 both have a thickness of 0.33 mm) represented by the mark “o” in FIG. 6 is assumed as the sample of the comparative example having an overall thickness of 0.66 mm. As is apparent from FIG. 6, even though Sample 1 of the present embodiment and Imaginary Sample 7 of the comparative example have the same thickness, the value of the ΔV differential/V1 for Sample 1 is greater than that for Imaginary Sample 7. In other words, Sample 1 of the present embodiment has a higher sensitivity than Imaginary Sample 7 of the comparative example even though both are of a similar strength.

Next, Sample 2 (present embodiment) having an overall thickness of 0.86 mm and a sample of the comparative example having an overall thickness of 0.86 mm are compared. For this comparison, Imaginary Sample 8 (wherein the first and second insulating layers 111 and 112 both have a thickness of 0.43 mm) represented by the mark “o” in FIG. 6 is assumed to be the sample of the comparative example having an overall thickness of 0.86 mm. As is apparent from FIG. 6, even though Sample 2 of the present embodiment and Imaginary Sample 8 of the comparative example have the same thickness, the value of ΔV differential/V1 for Sample 2 is greater than that for Imaginary Sample 8. Thus, Sample 2 of the present embodiment has a higher sensitivity than Imaginary Sample 8 of the comparative example even though both are of a similar strength.

Furthermore, Sample 3 (present embodiment) having an overall thickness of 1.07 mm and a sample of the comparative example having an overall thickness of 1.07 mm are compared. For this comparison, Imaginary Sample 9 (wherein the first and second insulating layers 111 and 112 both have a thickness of 0.535 mm) represented by the mark “o” in FIG. 6 is assumed to be the sample of the comparative example having an overall thickness of 1.07 mm. As is apparent from FIG. 6, even though Sample 3 of the present embodiment and Imaginary Sample 9 of the comparative example are of the same thickness, the value of ΔV differential/V1 for Sample 3 is greater than that for Imaginary Sample 9. Thus, Sample 3 of the present embodiment has a higher sensitivity than Imaginary Sample 9 of the comparative example even though both are of a similar strength.

The above results reveal that, for a given overall thickness of an element, the liquid-condition detection element 100 in which the first ceramic insulating layer 111 is thinner than the second ceramic insulating layer 112 has better sensitivity than an element in which the first and second ceramic insulating layers 111 and 112 have the same thickness. In other words, for a given overall thickness of the detection element, as compared with a liquid-condition detection element in which the first and second ceramic insulating layers have the same thickness, the liquid-condition detection element 100 of the present embodiment exhibits better sensitivity while having similar strength. Therefore, the liquid-condition detection element 100 of the present embodiment exhibits good sensitivity while having appropriate strength.

While the present invention has been described with reference to one preferred embodiment, the present invention is not limited thereto, but may be modified without departing from the spirit or scope of the invention.

For example, in the liquid-condition detection sensor 100, the outer, tubular electrode 10 and the inner, tubular electrode 20 are provided for detecting the level of the urea aqueous solution L. However, the outer, tubular electrode 10 and the inner, tubular electrode 20 may be omitted. In this case, the aforementioned detection of an abnormality of the urea aqueous solution L being absent is preferred.

In the liquid-condition detection sensor 100, the conductive layer 118 including the heat-generating resistor 117 is formed from a material which contains Pt as a main component. However, the material for the conductor layer 118 is not limited thereto, but may contain W, Mo, or the like as a main component. Also, the conductor layer 118 may contain a trace amount of a ceramic component (alumina in the present embodiment) used to form the first and second ceramic insulating layers 111 and 112.

Although the invention has been described above in relation to preferred embodiments thereof, it will be understood by those skilled in the art that variations and modifications can be effected in these preferred embodiments without departing from the scope and spirit of the invention.

What is claimed:

1. A liquid-condition detection element comprising:
a ceramic substrate of a laminated structure comprising a first ceramic insulating layer and a second ceramic insulating layer; and
a heat-generating resistor sealed in a liquid tight manner between the first ceramic insulating layer and the second ceramic insulating layer and having a resistance varying with the temperature thereof;
the liquid-condition detection element being immersed, in use, in liquid and the heat-generating resistor, in use, outputting an output signal related to a condition of the liquid in response to the heat-generating resistor being energized;
wherein the first ceramic insulating layer is of a thickness smaller than that of the second ceramic insulating layer.

2. A liquid-condition detection element according to claim 1, wherein an outer surface of the ceramic substrate includes a contact zone which, in use, comes into contact with the liquid.

3. A liquid-condition detection element according to claim 1, wherein the first ceramic insulating layer and the second ceramic insulating layer are of the same material.

4. A liquid-condition detection element according to claim 1, wherein the first ceramic insulating layer and the second ceramic insulating layer are formed by simultaneous firing.

5. A liquid-condition detection element according to claim 1, wherein the first ceramic insulating layer has a thickness and wherein connection conductors electrically communicating with the heat-generating resistor extend through the first ceramic insulating layer across thickness thereof, and wherein the output signal is output through the connection conductors.

6. A liquid-condition detection element according to claim 1, wherein the liquid is a urea aqueous solution.

7. A liquid-condition detection sensor comprising:
a liquid-condition detection element comprising:
a ceramic substrate of a laminated structure comprising a first ceramic insulating layer and a second ceramic insulating layer; and
a heat-generating resistor sealed in a liquid tight manner between the first ceramic insulating layer and the second ceramic insulating layer and having a resistance varying with the temperature thereof;
the liquid-condition detection element being immersed, in use, in liquid; and
a detection section for detecting a condition of the liquid on the basis of an output signal in accordance with the resistance of the heat-generating resistor produced by the heat-generating resistor when energized;
wherein the first ceramic insulating layer is of a thickness smaller than that of the second ceramic insulating layer.

8. A liquid-condition detection sensor according to claim 7, wherein an outer surface of the ceramic substrate of the liquid-condition detection element includes a contact zone which, in use, comes into contact with the liquid.

9. A liquid-condition detection sensor according to claim 7, wherein the first ceramic insulating layer and the second ceramic insulating layer of the liquid-condition detection element are of the same material.

10. A liquid-condition detection sensor according to claim 7, wherein the first ceramic insulating layer and the second ceramic insulating layer of the liquid-condition detection element are formed by simultaneous firing.

11. A liquid-condition detection sensor according to claim 7, wherein connection conductors of the liquid-condition detection element electrically communicate with the heat-generating resistor and extend through the first ceramic insulating layer across a thickness thereof and the output signal is output through the connection conductors.

12. A liquid-condition detection sensor according to claim 7, wherein the detection section energizes the heat-generating resistor for a predetermined period of time, obtains a first correspondence value and a second correspondence value corresponding to respective resistances of the heat-generating resistor at different times within the predetermined period of time, and determines at least a concentration of a particular component in the liquid on the basis of the first correspondence value and the second correspondence value.

13. A liquid-condition detection sensor according to claim 12, further comprising a level detector having a first electrode and a second electrode, the first and second electrodes forming a capacitor the capacitance of which varies with a level of the liquid,
wherein the liquid-condition detection element is integrated, in an insulated manner, with the level detector.

14. A liquid-condition detection sensor according to claim 7, wherein the liquid comprises a urea aqueous solution.
